(19) United States
(12) Patent Application Publication
(10) Pub. No.: US 2002/0114730 A1
(43) Pub. Date: Aug. 22, 2002

(54) Lemon extract and treatment methods

(76) Inventor: Darlene Jazzar, Sutton (CA)

Correspondence Address: Eric Fincham, 316 Knowlton Road, Lac Brome, QC, J0E 1V0, CA

(21) Appl. No.: 09/881,260

(22) Filed: Jun. 14, 2001

(30) Foreign Application Priority Data
Dec. 6, 2000 (CA) 2,327,738

(51) Int. Cl. A61L 2/18; A01N 65/00

(52) U.S. Cl. 422/28; 424/736

(57) ABSTRACT

A method for disinfecting a material which comprises applying to the place to be treated an effective amount of a lemon seed extract which is formed from lemon pulp and lemon seeds and optionally includes lemon rind and the pericarp thereof. The lemon seed extract is preferably in the form of a liquid emulsion and may be applied at a rate of between 50 to 150,000 ppm.

### FIELD OF THE INVENTION

The present invention relates to an extract derived from lemons and more particularly, relates to methods of disinfection and treatment using a lemon extract.

### BACKGROUND OF THE INVENTION

The use of disinfectants is well known in the art. This practice has become wide spread and some disinfectants are used in products as diverse as kitchen sponges, soap, fabrics and plastic. However, many people knowledgeable in the art are leery of such wide use of disinfectants. For example, it is known that triclosan, a widely used disinfectant, can force the development of “super bugs” that it cannot kill. It has been shown that changing just one gene in the E. coli bacterium allows it to resist triclosan. Triclosan has been widely used because it is what is known as a non specific biocide—it kills all microbes. It was believed to interrupt so many cell processes that it was not thought that an organism could develop a resistance to it. This is in comparison to many drugs which are used as antibiotics which work on a single process—i.e. penicillin stops many bacteria from building a strong cell wall by acting against one component known as a mucopeptide. Such a specific action means that many bacteria, including staphylococcus can resist penicillin.

The more a drug is used, the more chances bacteria have to evolve a resistance. Antibiotic resistant bacteria are becoming a bigger and bigger problem and the industry is developing stronger antibiotics to try and keep up with the resistance. There are now many people advocating that doctors cut back on frequent prescriptions of antibiotics except for those who really need them. Patients are also urged to take their full course of drugs to make sure no resistant bacteria survive to breed other resistant bacteria.

The use of antibiotics on an indiscriminate basis is gradually being understood to be a poor practice. Commonly, antibiotics are fed to healthy animals such as beef cattle and chickens in order to prevent infection. However, in the healthy intestinal track, dozens of different species of bacteria compete for nutrients. The ability of beneficial bacteria to maintain their positive balance of power over more harmful strains results in proper digestion and good health. Antibiotic use upsets this balance as it can destroy both good and bad bacteria. It is further being recognized that it is almost impossible to destroy 100% of the pathogenic bacteria without following the complete prescription period which, as is well known, many patients do not complete. Leaving behind even some of the harmful pathogens can have disastrous effects as now, without competition, these resistant mutants can double in population every few minutes causing re-infection with a resistant bacterial strain. As a result, there have been proposals to limit the use of antibiotics in animals.

### SUMMARY OF THE INVENTION

It is an object of the present invention to provide a lemon extract which is useful as a viricide, bacteriacide, and fungicide.

It is a further object of the present invention to provide a method for the treatment of animals for an undesired condition, either internal or external.

It is a further object of the present invention to provide a method for preservation of animal feed.

It is a further object of the present invention to provide a method for the disinfection of a surface.

It is a further object of the present invention to provide a method for the treatment of waste water.

It is a further object of the present invention to provide a method for the treatment of pool or spa water.

It is a further object of the present invention to provide a method for the treatment of well water.

It is a further object of the present invention to provide a method for the treatment of a feed processing room.

It is a further object of the present invention to provide a novel dental rinse and gargle solution.

According to one aspect of the present invention, there is provided a method for treating a material comprising the step of applying to a locus to be treated an effective amount of a lemon seed extract, the lemon seed extract being an extract from lemon pulp and lemon seeds.

According to a further aspect of the present invention, there is provided a method for the manufacture of a lemon seed extract comprising the steps of drying pulp and seeds from lemons, grinding the product to form a powder, washing the powder to remove inert material therefrom, drying the product to form a powder, and adding a liquid to the powder to form an emulsion.

The preparation of the lemon extract of the present invention is preferably formed from the normally waste by-products of a process of extracting lemon juice. The lemon extract is comprised of at least the pulp and seeds of the lemon and will be referred to herein as a lemon seed extract. Preferably, the lemon seed extract will include the rind and pericarp.

Although there are many ways of preparing the extract, according to one preferred method, the pulp and seeds of the lemon, which are obtained as a by-product of the juice extraction process, are dried and ground into a fine powder. Subsequently, the powder is dissolved in purified water and then the fiber and pectin are removed. The slurry is then spray dried at low temperatures forming a concentrated bioflavonoid powder.

Subsequently, the concentrated powder is dissolved in a distilled water and heated. Preferably, in an optional step, food grade ascorbic acid is added and the mixture is heated under pressure. One or more catalysts selected from a group including hydrochloric acid and natural enzymes may be added so that the material undergoes a catalytic conversion. The resulting slurry is skimmed and filtered, cooled and then treated with ultraviolet light.

The resulting product has ascorbic acid present in a range of between 25 mg/g and 30 mg/g. There is no trace of hydrochloric acid.

The pulp and seeds, as a by-product of the extracted juice industry, is a liquid which contains many polyphenolic compounds. The polyphenols themselves are unstable, but are converted into more stable substances which belong to the group of compounds known as quaternary ammonium compounds.

The final product is a very safe one. It is relatively stable and only the standard safety precautions need to be taken. It is generally not a skin irritant although it can be an irritant to the eyes and mucous membranes.

It is to be understood that the manner of functioning and possible mechanisms involved with the use of a lemon extract is based on an incomplete understanding thereof. Accordingly, any explanations given herein are advanced as possible theories for some of the mechanisms. It will, however, be understood that the invention is not limited to the theories herein described which are given only by way of a possible explanation of the results achieved. The term “lemon seed extract” is used to designate the product derived from the lemon pulp and seeds (and optionally the pericarp and rind).

The lemon seed extract is made by first converting lemon seeds and pulp into an acidic liquid.

This liquid is loaded with polyphenolic compounds, including quercitin, helperidin, campherol glycoside, neohelperidin, naringin, apigenin, rutinoside, poncirin, etc. The polyphenols themselves are unstable but are chemically converted into more stable substances that belong to a diverse class of products called quaternary ammonium compounds.

Some quaternary compounds, benzethonium chloride and benzalkonium chloride, for example, are used industrially as antimicrobials, but are toxic to animal life. The B vitamin choline is also a quaternary compound, but is non-toxic and even essential for maintaining healthy neurological function and fat metabolism.

Lemon seed extract features the best of both worlds: the quaternary compounds derived from the lemon fruit exhibit broad-spectrum antimicrobial activity, while evidencing none of the toxic side effects of chemically-derived quaternaries.

The finished product is a viscous, yellow-amber colored liquid that features a taste that is both bitter and acidic. Pure distilled water is added and a skimming process to remove excess fiber is done to reduce the bitterness and acidity to a lower level and to reduce the possibility that incidental contact could cause irritation to the skin or mucous membranes.

Studies indicate that the antimicrobial activity of lemon seed extract is in the cytoplasmic membrane where the uptake of amino acids is prevented and disorganization of the cytoplasmic membrane and leakage of low molecular-weight cellular contents occurs.

The uses of the extract according to the present invention are numerous. It can be used to treat humans and other animals including cats, dogs, cattle, horses, hogs, fish, poultry, etc. It can be used for the treatment of skin diseases, external injuries, fungal infections and internally, conditions caused by parasitic, bacterial, viral or fungal infections.

Generally, for external use, one can use a solution containing between 50 to 10,000 ppm of the concentrated extract. For internal use, preferably the concentrated extract is present in the solution between 15 and 500 ppm.

For skin fungi, parasitic or bacterial diseases of the skin, a solution of between 100 and 200 ppm would be placed on a skin or fur for a few minutes and then rinsed. The solution could also be used for hoof care on animals and the like.

Examples of some of the uses and suggested concentrations for the lemon seed extract are given in the table below.

TABLE 1

| | | | Drops per | |
|---|---|---|---|---|
| PPM | One Ounce(30 ml) | One Drop | Gallon (4.L.) | |
| (parts per | of Concentrate | of Concentrate | of Concentrate | Common Applications, |
| million) | in: | in: | in: | Suggested Uses: |
| 1,000-30,000 | One Gallon | One-half Tsp. | 1250 | Cosmetics. |
| | (3.78 liters) | (3 ml.) | | Surgical equipment. |
| | | | | Healing of Skin infections, burns, |
| | | | | wounds. |
| 500-3,000 | Ten Gallons | One ounce | 120-130 | Disinfectant & preservative for |
| | (37 liters) | (30 ml.) | | fresh fish, poultry, meat. |
| | | | | Cosmetics. |
| | | | | Mold. |
| | | | | Final rinse for fruits & vegetables |
| | | | | Surface disinfectant. |
| 100-2,000 | 20 Gallons | Two ounces | 60-65 | Disinfectant & preservative for |
| | (75 liters) | (60 ml.) | | fish, poultry, meat, fruits & |
| | | | | vegetables. |
| | | | | Add to water for Cut Flowers. |
| | | | | Animal feed additive. |
| | | | | General surface disinfectant. |
| | | | | Soap making. Mold. |
| 50-1,500 | 40 Gallons | Four ounces | 30-32 | Atomize cut flowers. |
| | (150 liters) | (120 ml.) | | Animal feed additive. |
| | | | | Water treatment. |
| | | | | Algae. |
| | | | | Mold. |
| | | | | Food preservative & antioxidant. |
| | | | | Crop spray. |
| 20-500 | 100 Gallons | Ten ounces | 10-12 | Ice Additive (fish case). |
| | (378 liters) | (300 ml.) | | Crop spray. |
| | | | | Animal feed additive. |
| | | | | Mold. |
| | | | | Algae. |
| | | | | Water treatment. |
| | | | | Food preservative |
| 10-250 | 200 Gallons | Twenty | 5-6 | Water Treatment. |
| | (756 liters) | ounces | | Crop spray. |
| | | (600 ml.) | | Food preservative. |

The product, as above mentioned, is not irritating to skin although if undiluted on the tongue, could cause irritation. It is non-corrosive to metal, wood, fabric, plastic, painted surfaces, etc. Although it is a powerful anti-oxidant, it is non-toxic, non-volatile and does not alter the smell or flavor of produce or other foods in normal dilutions. It could be applied right up until harvest and through the shipping, handling and storage phases without concern. It can be used with people and/or animals present in the area. There is no build-up in the soil. Any residue on application tools is actually a desirable attribute. The product is biodegradable and does not pollute. It should be noted, however, that extremely hard water or water with high levels of chlorine may require an increased concentration for effective results.

The product can be applied by spray (from the ground or the air) or can be metered into the water supply for greenhouses and hydroponic operations. When making large batches, for maximum effectiveness, it is important to obtain a homogeneous mixture of the product. It is therefore recommended to make a pre-mixture in a lesser quantity of liquid, and then admix with the rest of the liquid. Liquid or powdered product can be mixed into seedbeds, potting soil, mulch, fertilizer, and compost. When watering houseplants or small lots, 5 to 10 drops of the product per gallon are sufficient to keep most pathogens at bay.

The product is most effective against microscopic organisms, but can also be effective against larger farm and garden pests. This is true especially of soft-bodies pests such as aphids and slugs. Japanese beetles and other hard-shelled bugs are not as easily inhibited, but they do respond. A combination of the product and cayenne pepper can be especially effective, and even more so when combined with garlic. The product can be combined with numerous other agents, as indicated, without any known “interactivities” or “contraindications”. The product is biodegradable, so concern for earthworms and beneficial bacteria are minimal. (In humans and animals, the product does not disturb the beneficial bacteria in the intestines with normal dosages).

The product mixes well with virtually all solvents, including water, alcohol, and organic solvents.

Having thus generally described the invention, reference will be made to accompanying examples illustrating embodiments of the invention.

### EXAMPLE 1

In this example, Listeria Monocytogenes ATCC #15313 were exposed to the product set forth above. The results were as follows:

| Organism | Control | 1 Minute | 2 Minutes | 5 Minutes |
|---|---|---|---|---|
| Listeria | cfu/mL | CCU/ml | CCU/ml | CCU/ml |
| Monocytogenes | | | | |
| ATCC #15313 | | | | |
| | 12.4 × 10⁵ | <10 | <10 | <10 |
| % Log Reduction | | >6.09 | >6.09 | >6.09 |

### EXAMPLE 2

| Organism | Control | 1 Minute | 2 Minutes | 5 Minutes |
|---|---|---|---|---|
| Campylobacter | cfu/mL | cfu/mL | cfu/mL | cfu/mL |
| jejuni | | | | |
| ATOC #29428 | 1.53 10⁶ | <10 | <10 | <10 |
| % Log Reduction | — | >6.18 | >6.18 | >6.18 |

### EXAMPLE 3

The microorganisms, Escherichia coli, Staphylococcus aureus, Salmonella arizonae, at a concentration of approximately one million CCU, were placed in a container containing 10 ml of the product. After 120 seconds, samples of each were tested and there was no live colonies left.

### EXAMPLE 4

The microorganisms, Staphylococcus aureus, Staphylococcus epidermitis, Candida albicans, each at a concentration of approximately one million CCU, were placed individually in containers having 10 ml of the product at a concentration of 0.16%. After 120 seconds, an aquelot was tested and each instance, there was no microbial activity present.

### EXAMPLE 5

In this example, a number of different gram-negative, gram-positive, fungi, yeasts, and other organisms were tested to find the minimum inhibitory concentration in-vitro (MIC), and the minimum inhibitory concentration is the amount to totally kill the organism and assure no re-growth thereof. As will be noted, the MIC will vary substantially depending upon the particular organism.

| | Origin | Strain # | MIC (ppm) |
|---|---|---|---|
| Gram-negative Bacteria | | | |
| Aerobacter aerogenes | CITM | 413 | 20 |
| Alcalingenes faecalis | A | | 2000 |
| Brucell intermedia | A | | 2 |
| Brucella abortus | NCTC | 8626 | 2 |
| Brucella melitensis | A | | 2 |
| Brucella suis | A | | 2 |
| Cloaca cloacae | NCTC | 8155 | 6 |
| Eseherichia coli | NCTC | 86 | 2 |
| Escherichia coli | ATCC | 9663 | 6 |
| Escherichia coli | NCTC | 9001 | 6 |
| Haemophulus influenzae | A | | 660 |
| Klebsiella edwardsii | NCTC | 7242 | 6 |
| Klebs. aerogenes | NCTC | 8172 | 6 |
| Klebs. pneumoniae | ATCC | 4352 | 6 |
| Legionella pneumoniae | Isolate | | 200 |
| Loefflerella mallei | NCTC | 9674 | 6 |
| Loeff. pseudomallei | NCIB | 10230 | 20 |
| Moraxella duplex | A | | 2 |
| Moraxella glucidolytica | A | | 6 |
| Neisseria catarrhalis | NCTC | 3622 | 660 |
| Pseudomonas capacia | C-175 | | 5000 |
| Pasteurella septica | NCTC | 948 | 2 |
| Past. pseudotuberculosis | C. - G. | | 200 |
| Proteus vulgaris | NCTC | 8313 | 2 |
| Proteus mirabilis | A | | 6 |
| Pseudomonas aeruginosa | NCTC | 1999 | 2000 |
| Pseudom. aeruginosa | ATCC | 12055 | 20,000 |
| Pseudom. aluorescens | NCTC | 4755 | 2000 |
| Salmonella choleraesuis | | | 50 |
| Salmonella enteritidis | A | | 6 |
| Salm. gallinarum | | | 50 |
| Salm. typhimurium | NCTC | 5710 | 6 |
| Salm. typhi | NCTC | 8384 | 6 |
| Salm. paratyphi A | NCTC | 5322 | 6 |
| Salm. paratyphi B | NCTC | 3176 | 6 |
| Salm. pullorum | ATCC | 9120 | 6 |
| Serratia marcescens | A | | 2000 |
| Shigella flexneri | NCTC | 8192 | 6 |
| Shigella sonnei | NCTC | 7240 | 3 |
| Shigella dysenteriae | NCTC | 2249 | 2 |
| Vibro Cholerae | A | | 200 |
| Vibro eltor | NCTC | 8457 | 200 |
| Gram-positive Bacteria | | | |
| Bacillus subtilis | NCTC | 8236 | 2 |
| Bacillus megatherium | A | | 60 |
| Bacillus cereus | A | | 60 |
| Bacillus cereus var. mycoides | A | | 60 |
| Clostridium botulinum | NCTC | 3805 | 60 |
| Clostridium tetani | NCTC | 9571 | 60 |
| Corynebacterium acnes | ATCC | 6919 | 60 |
| Corynebacterium diphteriae | ATCC | 6917 | 60 |
| Coryn. diphtheriae | A | | 60 |
| Coryn. minutissium | ATCC | 6501 | 100 |
| Diplococcus pneumoniae | NCTC | 7465 | 60 |
| Lactobacillus arabinosus | CITM | 707 | 66 |
| Lactob. arabinosus | ATCC | 8014 | 66 |
| Lactobacillus casei | CITM | 707 | 100 |
| Listeria monocytogenes | ATCC | 15313 | 20 |
| Mycobacterium tuberculosis | A | | 2000 |
| Mycobacterium smegmatis | NCTC | 8152 | 20 |
| Mycobacterium phlei | A | | 6 |
| Sarcina lutea | NCTC | 196 | 60 |
| Sarcina ureae | ATCC | 6473 | 2 |
| Staphylococcus aureas | NCTC | 7447 | 2 |
| Staph. aureas | NCTC | 4163 | 2 |
| Staph. aureas | NCTC | 6571 | 6 |
| Staph. aureas | NCTC | 6966 | 2 |
| Staph. aureas | ATCC | 13709 | 2 |
| Staph. aureas | ATCC | 6538 | 2 |
| Staph. albus | NCTC | 7292 | 2 |
| Staph. albus | C. - G. | | 6 |
| Streptococcus agalactiae | NCTC | 8181 | 60 |
| Strep. haemoyticus A | A | | 20 |
| Strep. faecalis | NCTC | 8619 | 200 |
| Strep. faecalis | ACTCC | 10541 | 60 |
| Strep. pyogenes | NCTC | 8322 | 60 |
| Streptococcus viridans | | | 20 |
| Fungi & Yeasts | | | |
| Aspergillus niger | ATCC | 6275 | 600 |
| Aspergillus fumigatus | ATCC | 9197 | 200 |
| Candida albicans | A | | 60 |
| Candida albicans | ATCC | 10259 | 60 |
| Epidermophyton floccosum | ATCC | 10227 | 200 |
| Keratinomyces ajelloi | A | | 200 |
| Monilia albicans | | | 10 |
| Saccharomyces cerevisiae | | | 60 |
| Trichophyton mentagrophytes | ATCC | 9533 | 20 |
| Trichophyton rubrum | A | | 200 |
| Trichophyton tonsurans | A | | 200 |

As will be seen from the above, the lemon seed extract is effective against a number of different types of organisms.

The present invention can be used in a number of different applications. As previously mentionned, it can be used to treat any product wherein it is desired to disinfect a surface. This can range from its use as a substitution for everyday cleaners which are used in household tasks to its use as an industrial non-specific biocide.

The present invention can be used in a number of methods involving life. Thus, as previously discussed, it could be used to treat all types of livestock when their meat is being prepared for human consumption. The lemon seed extract can be used at many different points in the chain of preparation. Thus, it can be used in the slaughterhouse, in the meat packing plant, and at the retail level.

Still further, the lemon seed extract of the present invention can be used as an additive to feed for the animals. In so doing, the lemon seed extract could function both as a preservative for the feed and it is believed also have beneficial effects for the animal.

The lemon seed extract may also be used in the treatment of water including both well water and wastewater. In the treatment of well water, the lemon seed extract may be used either alone or in a combination with other known treatments. In the treatment of wastewater, it may be utilized as one of the final steps in a multi-step process.

It will be understood that the above described embodiments are for purposes of illustration only and that changes and modifications may be made thereto without departing from the scope and spirit of the invention. Thus, though some of the uses have been described above, the method can be practiced wherever it is desired to have an agent work as a bacteriacide, viricide or fungicide. Thus, some of the uses are given in Table 1 of this document, these uses are not intended to be limiting, but being merely illustrative thereof.

I claim:

1. A method for treating a material comprising the step of applying to a locus to be treated an effective amount of a lemon seed extract, said lemon seed extract being an extract from lemon pulp and lemon seeds.

2. The method of claim 1 wherein said lemon seed extract further includes lemon rind and pericarp.

3. The method of claim 2 wherein said lemon seed extract is a powder, said powder being formed when said lemon pulp and lemon seeds are dried, ground, washed and then dehydrated.

4. The method of claim 2 wherein said lemon seed extract comprises a liquid emulsion, said liquid emulsion being formed when said lemon pulp and lemon seeds are dried, ground, washed, dehydrated and then a liquid added to form said emulsion.

5. The method of claim 1 wherein said material uses pool water, said lemon seed extract being applied at a rate of between 50 to 10,000 ppm.

6. The method of claim 1 wherein said lemon seed extract is applied in a feed processing plant.

7. The method of claim 1 wherein said lemon seed extract is added to well water.

8. The method of claim 1 wherein said lemon seed extract is added to animal feed.

9. The method of claim 1 wherein said lemon seed extract is added to a dental rinse.

10. The method of claim 1 wherein said lemon seed extract is applied to an external surface of a mammal.

11. The method of claim 1 wherein said lemon seed extract is added to drinking water.

12. A method for the manufacture of a lemon seed extract comprising the steps of:
a) drying pulp and seeds from lemons;
b) grinding the product from step (a) to form a powder;
c) washing said powder to remove inert material therefrom;
d) drying the product from step (c) to form a powder; and
e) adding a liquid to said powder from step (d) to form an emulsion.

13. The method of claim 12 wherein step (a) includes the step of drying lemon rind and lemon pericarp with said pulp and seeds.

14. The method of claim 13 wherein step (c) comprises washing said powder with HCl and absorbic acid.

15. The method of claim 13 further including the step of adding to the powder from step (d) by citrus bioflavonoids.

16. A lemon seed extract suitable for use as a bacteriacide, the lemon seed extract being produced by the method of claim 4.
